(19) United States
(12) Patent Application Publication
(10) Pub. No.: US 2015/0238100 A1
(43) Pub. Date: Aug. 27, 2015

(54) SENSOR ELECTRODE DEVICE

(71) Applicant: NATIONAL CHIAO TUNG UNIVERSITY, HSINCHU CITY (TW)

(72) Inventors: CHIN-TENG LIN, HSINCHU COUNTY (TW); JUI-HUANG CHENG, NEW TAIPEI CITY (TW); SHAO-WEI LU, HSINCHU CITY (TW)

(73) Assignee: NATIONAL CHIAO TUNG UNIVERSITY, HSINCHU CITY (TW)

(21) Appl. No.: 14/191,673

(22) Filed: Feb. 27, 2014

(51) Int. Cl. A61B 5/04; A61B 5/0492; A61B 5/0496; A61B 5/0478; A61B 5/0408

(52) U.S. Cl. CPC A61B 5/04; A61B 5/0478; A61B 5/0408; A61B 5/0496; A61B 5/0492

(57) ABSTRACT

A sensor electrode device comprises a base and a plurality of flexible electrodes. The flexible electrodes protrude from one side of the base, detecting at least one physiological signal and sending out the physiological signal via the base. The flexible electrodes can expel hairs to directly contact the skin, whereby hairs would not hinder the sensor electrode device from detecting physiological signals. Further, the tips of the flexible electrodes bend under pressure to increase the area contacting the skin and improve the ability to detect physiological signals.

### BACKGROUND OF THE INVENTION

1. Field of the Invention

The present invention relates to an electrode technology, particularly to a flexible sensor electrode device.

2. Description of the Related Art

Many secrets of human bodies have been compromised by modern medical technologies, and the physician can learn states of a patient via detecting the physiological signals. For example, the physician can detect the abnormalities of the heart according to ECG (electrocardiogram)—a record of electrical activities of the heart; the physician can diagnose the diseases of the muscle, neural system or even cerebellum according to EMG (electromyogram)—a record of the electrical potential generated by muscle cells; the physician can evaluate the states of consciousness, emotions, coma, apoplexy, epilepsy, encephalitis, and other encephalitic diseases according to EEG (electroencephalogram)—a record of voltage fluctuations resulting from ionic current flows within the neurons of the brain.

At present, most physiological signal measurements adopt wet electrodes. Wet electrodes are stuck to the skin, and then clipped by metallic clampers for detecting physiological signals, such as EEG signals. For traditional EEG recording, at least 16 channels of wet sensors are applied to the scalp. Conductive gel is injected between the metallic clampers and the scalp to maintain as low impedance as <50 Ohm to acquire well-defined EEG signal. However, wet electrodes consume much time to prepare and may wound the head skin. Besides, conductive paste will be dried about half an hour later and needs replenishing each half an hour. Thus, the head of the testee is usually covered by a lot of conductive paste. Currently, there is also a dry electrode, which is a cube having less than 1.5 cm in width and length and exempted from using conductive paste. However, measurement is still interfered with by hair when this type of dry electrode is applied to measuring brain waves.

Accordingly, the present invention proposes a sensor electrode device to overcome the abovementioned problems.

### SUMMARY OF THE INVENTION

The primary objective of the present invention is to provide a sensor electrode device, whose flexible electrodes can harmlessly expel hair to directly contact head skin and detect brain waves without hair razoring or conductive paste application.

Another objective of the present invention is to provide a sensor electrode device, whose base and flexible electrodes are full-body electrically conductive, and whose flexible electrodes do not necessarily contact the skin only at their tips but can bend to increase the area contacting the skin.

To achieve the abovementioned objectives, the present invention proposes a sensor electrode device, which comprises a base and a plurality of flexible electrodes. The flexible electrodes protrude from one surface of the base. The flexible electrodes detect a physiological signal and send out the physiological signal via the base. The flexible electrodes would not be hindered by hairs but can expel hairs to directly contact the skin for physiological measurement.

Below, embodiments are described in detail to make easily understood the objectives, technical contents, characteristics and accomplishments of the present invention.

### BRIEF DESCRIPTION OF THE DRAWINGS

FIG. 1 is a perspective view schematically showing a sensor electrode device according to one embodiment of the present invention;

FIG. 2 is a side view of the sensor electrode device shown in FIG. 1; and

FIG. 3 is a perspective view schematically showing a sensor electrode device according to another embodiment of the present invention.

### DETAILED DESCRIPTION OF THE INVENTION

The present invention discloses a sensor electrode device, which is applied to a brain-wave cap for detecting brain waves or attached to the chest for detecting electrocardiographic signals.

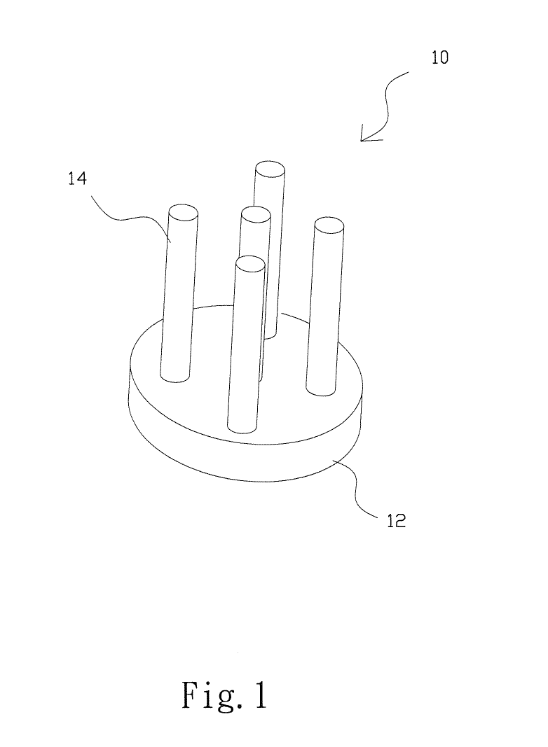

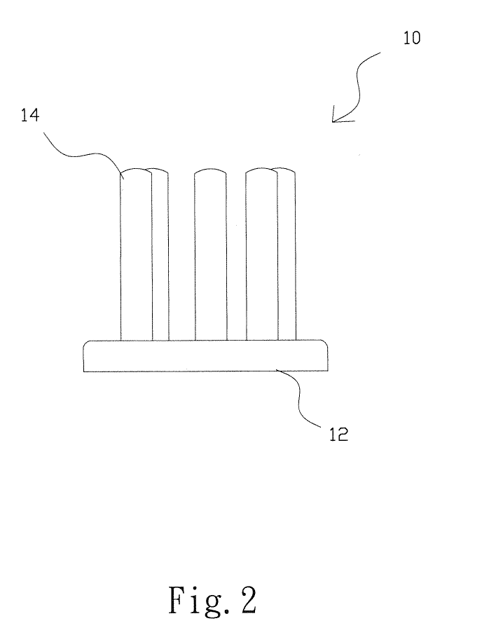

Refer to FIG. 1 and FIG. 2 respectively a perspective view and a side view of a sensor electrode device according to one embodiment of the present invention. The sensor electrode device 10 of the present invention comprises a base 12 and a plurality of flexible electrodes 14. The base 12 may have an arbitrary shape. In one embodiment, the base 12 has a circular shape. The flexible electrodes 14 protrude vertically from one side of the base 12, detect at least one physiological signal, and send out the physiological signal via the base 12. The present invention does not constrain the number, length and thickness of the flexible electrodes 14. In the embodiment shown in FIG. 1 and FIG. 2, the sensor electrode device 10 contains 5 flexible electrodes 14 having an identical length and an identical thickness.

The contact area, impedance, conductivity, etc. influence the performance that the electrodes receive physiological signals. The base 12 and the flexible electrodes 14 are made of a flexible and conductive material, such as silver plated glass-embedded silicone. The base 12 and the flexible electrodes 14 are fabricated into a one-piece component in an injection-molding method or a hot-pressing method.

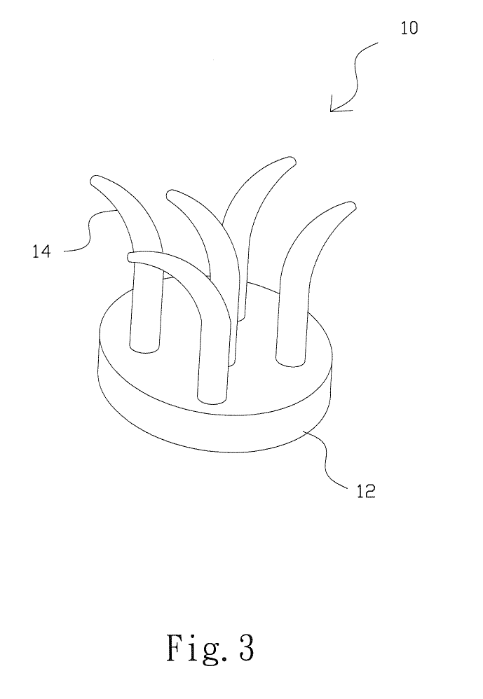

In the embodiment shown in FIG. 1 and FIG. 2, the flexible electrodes 14 are straight rods vertical to the base 12. In the embodiment shown in FIG. 3, the tails of the flexible electrodes 14 are outwardly and spirally curved to favor the flexible electrodes 14 to expel hairs.

The sensor electrode devices of the present invention may be arranged in a brain-wave cap or another physiological measurement instrument. After the flexible electrodes have contacted skin, the pressure will further make the tips of the flexible electrodes bend and expel the surrounding hairs to detect physiological signals, such as ECG signals, EEG signals, EOG signals, and EMC signals. Therefore, the present invention is exempted from the hindrance of hairs and able to transmit signals to the measurement instrument clearly and completely. Besides, the flexible electrodes would not wound skin.

In conclusion, the present invention proposes a sensor electrode device comprising a base and a plurality of electrodes, which are flexible and full-body electrically conductive. The flexible electrodes can expel hairs to directly contact skin and detect physiological signals from skin without hair razoring or conductive paste application. Further, the flexible electrodes can bend to increase the area contacting the skin and improve the ability to detect physiological signals.

The embodiments described above are only to exemplify the present invention but not to limit the scope of the present invention. Any equivalent modification or variation according to the characteristic or spirit of the present invention is to be also included within the scope of the present invention.

What is claimed is:

1. A sensor electrode device comprising
a base; and
a plurality of flexible electrodes protruding from one side of said base, detecting at least one physiological signal, and sending out said physiological signal via said base.

2. The sensor electrode device according to claim 1, wherein said base and said flexible electrodes are made of an electrically conductive material.

3. The sensor electrode device according to claim 1, wherein said base is made of a flexible material.

4. The sensor electrode device according to claim 1, wherein said base and said flexible electrodes are made of silver plated glass-embedded silicone.

5. The sensor electrode device according to claim 1, wherein said physiological signal is an EEG (electroencephalography) signal, an ECG (electrocardiography) signal, an EOG (electrooculography) signal or an EMG (electromyography) signal.

6. The sensor electrode device according to claim 1, wherein said base and said flexible electrodes are fabricated into a one-piece component.

7. The sensor electrode device according to claim 1, wherein said base and said flexible electrodes are fabricated with a hot-pressing method or an injection-molding method.

8. The sensor electrode device according to claim 1, wherein said flexible electrodes are with an angle to said base.

9. The sensor electrode device according to claim 1, wherein said flexible electrodes protrude from said base, and wherein tips of said flexible electrodes are curved outwardly and spirally.
